(19) United States
(12) Patent Application Publication
(10) Pub. No.: US 2011/0076251 A1
(43) Pub. Date: Mar. 31, 2011

(54) NOVEL THERAPEUTIC AGENTS AGAINST HEPATITIS

(76) Inventor: Joerg Friedrich Schlaak, Essen (DE)

(21) Appl. No.: 12/992,974

(22) PCT Filed: Mar. 12, 2009
(86) PCT No.: PCT/EP2009/001780

§ 371 Date: Nov. 16, 2010

(30) Foreign Application Priority Data
May 16, 2008 (DE) 10 2008 024 010.9
Jun. 24, 2008 (DE) 10 2008 029 669.4

(51) Int. Cl. A61K 38/21; A61K 31/713; C12Q 1/70; C12Q 1/68; C07H 21/02; A61P 31/12

(52) U.S. Cl. 424/85.4; 514/44 A; 435/5; 435/6; 536/24.5

(57) ABSTRACT

This invention relates to the treatment of hepatitis infections or hepatitis diseases, in particular hepatitis C. The invention more particularly relates to the use of an inhibitor and/or repressor of a nucleic acid molecule, especially gene, which is related to the proliferation and/or replication of hepatitis viruses, in particular hepatitis C viruses, in order to produce a medicament for preventing and/or curing hepatitis, in particular hepatitis C, as well as a pharmaceutical composition, preferably for preventing or treating hepatitis C diseases, said composition containing the repressor and/or inhibitor.

### CROSS-REFERENCES TO RELATED APPLICATIONS

This application is a National Stage filing of International Application PCT/EP 2009/001780, filed Mar. 12, 2009, entitled “NOVEL THERAPEUTIC AGENTS AGAINST HEPATITIS” claiming priority to German Applications Nos. DE 10 2008 024 010.9 filed May 16, 2008, and DE 10 2008 029 669.4 filed Jun. 24, 2008. The subject application claims priority to PCT/EP 2009/001780, and to German Application Nos. DE 10 2008 024 010.9, and DE 10 2008 029 669.4 and incorporates all by reference herein, in their entirety.

### BACKGROUND OF THE INVENTION

The present invention relates to the use of substances, in particular in the form of inhibitors or repressors, which are capable of regulating the gene activity of a gene associated with the multiplication or replication of hepatitis viruses, in particular hepatitis C viruses, in the field of the diagnosis and therapy of hepatitis, in particular hepatitis type C. The gene associated with the multiplication or replication of hepatitis C viruses is preferably the human interferon-stimulated gene ISG15.

The present invention furthermore relates to the use of a substance which neutralizes or inhibits or at least reduces the gene activity of a gene associated with the multiplication or replication of hepatitis viruses, in particular hepatitis C viruses, in particular of an inhibitor or repressor, for preparing a medicament or pharmaceutical for the prophylactic and/or curative treatment of hepatitis, in particular hepatitis type C.

In addition, the present invention relates to the use of an interferon-stimulated gene, in particular ISG15, for identifying and/or providing a pharmaceutical for the prophylactic and/or curative treatment of hepatitis, in particular hepatitis type C, and/or for predicting individual effects of pharmaceuticals and/or for predicting side effects of or the response to pharmaceuticals.

In addition, the present invention relates to a process for identifying substances, in particular inhibitors and/or repressors, which regulate the gene activity of an interferon-stimulated gene, in particular associated with the multiplication and/or replication of hepatitis viruses, in particular hepatitis C viruses, and to a process for identifying substances which regulate the activity of the corresponding gene products or of products associated with the gene. Furthermore, the present invention relates to a process for improving the pharmacological properties of these substances.

Finally, the present invention relates to a pharmaceutical composition comprising at least one pharmacologically active substance, in particular inhibitor or repressor, identified based, on the processes according to the invention, where the substance modulates, in particular inhibits, the gene activity of a gene associated with the multiplication or replication of hepatitis C viruses; moreover, the present invention relates to a pharmaceutical composition, preferably for the prophylactic or therapeutic treatment of hepatitis C disorders, which comprises effective, in particular pharmaceutically effective, amounts of at least one siRNA.

### BRIEF SUMMARY OF THE INVENTION

Chronic infection with the hepatitis C virus (HCV) is, with about 170 million infected people world-wide, a global health problem. At a chronification rate of about 80%, hepatitis type C is one of the main causes of hepatitis, cirrhosis of the liver and liver cell carcinomas.

The hepatitis C virus belongs to the family of the Flaviviridae and is the only representative of the genus of the hepaci viruses. The hepatitis C virus was initially classified among the so-called NonA-NonB hepatitis viruses until, in 1989, the viral genome was sequenced. To date, six genotypes have been characterized which, for their part, are divided into subtypes. In most cases, infection, with the virus is via transfusion of infected banked blood, in particular in the seventies, or via injuries with syringes, for example among hospital personnel. Further possible ways of transmission are unprotected sexual intercourse and the use of shared syringes among drug addicts. In most cases, acute infection is asymptomatic. In 70 to 80% of the cases, this is followed by a chronic infection of the liver which may progress to cirrhosis of the liver and hepatocellular carcinoma.

The prior art uses, as currently the most efficient therapy for chronic hepatitis type C, interferon (IFN), in particular α-interferon (synonymously also referred to as interferon-alpha or IFN-α), preferably pegylated IFN-α, if appropriate in combination with the virus static ribavirin. Depending on the genotype and other factors, this therapy results in a cure in only 50 to 90% of the patients. One of the most frequent side effects of this therapy is IFN-induced severe depression which, in addition to a worsening of the quality of life, may lead to a termination of the therapy or even to suicide. As yet, it has not been possible to develop a vaccine against the hepatitis C virus.

Interferons (IFN) are low-molecular-weight proteins and are classed with the cytokines. A distinction is made between interferons of type I and interferons of type II. The interferons of type I are present in monomeric form and can be divided into superfamilies, their main difference being their origin. From among the interferons of type I, IFN-α and IFN-β are most prevalent. The group of the α-interferons in turn can be divided into a number of subtypes expressed by different genes. They are mainly formed in leucocytes and fibroblasts, whereas IFN-β is produced mainly by endothelial cells and fibroblasts. IFN-λ and IFN-ω also belong to the interferons of type I.

Expression of the interferons of type I is induced by the recognition of viral, but also bacterial, pathogenic patterns. They are secreted by infected cells and act both parakrine onto neighboring cells and autokrine onto the IFN-producing cell itself. Receptor binding triggers a signal cascade terminating in the induction of interferon-stimulated genes (ISGs).

The activity of more than 150 ISGs is additionally increased by a modification, and their performance is enhanced. ISG15, an IFN-induced 15 kD protein, has a ubiquitin-like domain and is attached covalently to the target proteins via a set of enzymes (E1=Ube1L, E2=UbcH8 and E3=Herc5), as in ubiquitinylation. In the literature, this process is also referred to as ISGylation, The enzymes E1, E2 and E3 are likewise upregulated by type T interferons, leading to an increased ISGylation and enhancing the interferon response. However, at the same time, there is also an induction of Usp18, the protease which reverses ISGylation, which, in addition to shorter half-lives and with further negative regulation mechanisms, limits the time that IFN is active.

Further with respect to the interferons, these are, by virtue of their properties and the ability to induce a large number of cellular defense mechanisms and to support the endogenous immune response, used for the therapy of tumors, autoimmune disorders and virus infections (for example HBV and HCV).

As described above, in the therapy of hepatitis type C disorders or infections, the use of in particular pegylated IFN-α2a or IFN-α2b in combination with the nucleoside analog ribavirin (RBV) has become established. The response to the therapy depends on the HCV genotype, the virus load, the age of the patient, existing liver damage and coinfections. In infections with genotypes 2 and 3, up to 80% of the patients show a sustained decline of the virus after 24 weeks, whereas in infections with genotype 1 only about 50% of the patients respond to the therapy and have a sustained decline of the virus load after 48 weeks, at a body weight-dependent RBV dosage.

The response to the therapy can be divided into two phases. An early viral response (EVR) depending on the genotype and the medicament dose used in the first phase is followed by a slower but sustained viral response (SVR) in the second phase. Studies have shown that, if, within the first 12 weeks after initiation of the therapy, there is no EVR with a decline of the viral load by at least 2log₁₀ phases, a sustained response is not to be expected, and the therapy can be terminated. Different expression patterns of the ISGs both in PBMCs (peripheral blood mononuclear cells) and in liver biopsies of patients also allow prognoses to be made about a response to the therapy.

The abovementioned therapy of the prior art thus has the disadvantages of in some cases severe side effects, in particular with regard to the onset of depression, and the fact that an effective therapeutic success—as described above—is not always ensured.

Against this background, it is an object of the present invention to provide novel and efficient therapeutic and treatment options for hepatitis disorders, in particular hepatitis C disorders, which, compared to the approaches known from the prior art, are more effective and, at the same time, have reduced side effects.

In this context, it is the further object of the present invention to provide a process for identifying substances or substances as such which allow a reduction or neutralization of the gene expression or gene activity of genes associated with the multiplication or replication of hepatitis C viruses, in particular ISG15, in order to at least reduce virus multiplication or replication in this way.

Besides, it is a further object of the present invention to provide a process which allows the identification of specific genes which play a decisive part in the multiplication or replication of hepatitis C viruses, these preferably being human and/or interferon-stimulated genes.

Finally, one of the objects on which the invention is based is to provide processes, uses and substances of the type mentioned above which avoid the disadvantages of the prior art or else are capable of at least reducing or lessening them.

### BRIEF DESCRIPTION OF THE DRAWINGS

FIG. 1A shows the inhibition of the HCV replication and the lack of induction of resistant, mutants on. treatment with ISG15-specific siRNA (siISG15) compared to a treatment with siNC, where the HCV copy number has been normalized against the untreated control (MH1) and compared.

FIG. 1B shows the inhibition of the HCV replication and the lack of induction of resistant mutants on treatment with ISG15-specific siRNA (siISG15) compared to a treatment with siNC, where the HCV/ISG15 copy number has been normalized against the siNC control and compared.

FIG. 2A shows, by way of a Westernblot, the results of a treatment, carried out on human conl HCV replicon cells, with siRNA directed against ISG15, firstly without additional administration of IFN-α (−IFN-α) and secondly with additional administration of IFN-α (+IFN-α).

FIG. 2B shows, by way of a Westernblot, the results of a treatment, carried out on murine MH1 HCV replicon cells, with siRNA directed against ISG15, firstly without additional administration of IFN-α (−IFN-α) and secondly with additional administration of IFN-α (+IFN-α).

FIG. 3 shows a comparison of the replication of hepatitis C viruses in MH1 cells for various passages, where the HCV copy number is based on 100,000 β-aktin molecules (MH1=untreated control, NC=non-silencing control, ISG15=ISG15-specific siRNA). For all passages, when ISG15-specific siRNA is used, a marked decline of the HCV copy number and thus a reduction of virus synthesis and replication can be observed compared to MH1 and NC.

### DETAILED DESCRIPTION OF THE INVENTION

In the context of the present invention, the applicant was, in a completely surprising manner, able to identify, for the first time, a specific gene which is decisive in connection with the multiplication or replication of hepatitis C viruses in a host system, in particular in man. In this context, the applicant has found, in a completely surprising manner, that the interferon-stimulated gene ISG15 is decisive in the abovementioned multiplication or replication of hepatitis C viruses, and that induction or activation of this gene may result in an unwanted increase of the replication of the virus. The gene ISG15 identified with a view to its relevance regarding the multiplication or replication of hepatitis C viruses can thus be used according to the invention as a basis for processes and medicaments for the therapy of a hepatitis C disorder or infection where, in this respect, it is, in accordance with the invention, the aim to inactivate the gene in question.

Accordingly, the applicant has found, in a completely surprising manner, that the targeted use of an inhibitor or repressor with which the (gene) activity of the gene associated with the multiplication or replication of hepatitis C viruses, in particular ISG15, and thus a modulation of its gene activity leads to a significant reduction of the hepatitis C viral load, which can be attributed substantially to a markedly reduced multiplication or replication of the hepatitis C viruses. In this context, the applicant was—completely surprisingly—able to show in particular that the targeted use of a so-called siRNA specifically adapted to ISG15 or directed against ISG15 results, on its application, in a marked reduction of the gene activity of ISG15 and as a consequence in a significantly reduced synthesis or multiplication or replication of hepatitis C viruses in the host, system. In this manner, the applicant, was able to provide a completely novel therapeutic approach, which in addition has few side effects, and on the basis of which it is possible to treat a hepatitis C disorder effectively.

It goes without saying that developments, embodiments, advantages and the like which hereinbelow are specified for only one aspect of the invention, to avoid repetition, do, of course, also apply correspondingly to the other aspects of the invention.

Thus—according to a first aspect of the present invention—the present invention provides the use of an inhibitor and/or repressor of a nucleic acid molecule, in particular gene, associated with the multiplication and/or replication of hepatitis viruses, in particular hepatitis C viruses, and/or its DNA sequence and/or its assigned RNA sequence and/or its assigned (poly)peptide for preparing a pharmaceutical for the prophylactic and/or therapeutic treatment of hepatitis, in particular hepatitis type C.

As indicated above, the central idea of the present invention is thus the specific reduction and/or neutralization, in particular inhibition, of the gene activity of such a gene which is associated with the multiplication or replication or synthesis of hepatitis C viruses in particular in the respect that expression and/or activation of the gene which may be induced, for example, owing to the virus infection, leads to an increased replication or multiplication of hepatitis C viruses in the host.

In this context, the term “inhibitor” and/or “repressor” used in accordance with the invention relates in particular to a substance which reduces and/or neutralizes, in particular inhibits, the gene activity. In other words, the inhibitor or the repressor is a compound which neutralizes or at least reduces the gene activity, where the respective pharmacological activity of the inhibitor or repressor may be both on the level of the gene, i.e. for example by direct, interaction with the gene or with promoters and/or enhancers assigned thereto, and on the level of the gene product(s), such as transcription products (for example mRNA) and/or translation products (for example proteins). In addition, the pharmacological activity or the gene activity—reducing and/or—neutralizing, in particular—inhibiting activity of the inhibitor or repressor may act on the level of gene-regulated mediators or factors and/or on the level of gene-regulating mediators or factors. Thus, the inhibitor or repressor used according to the invention may result in a reduction of the expression or gene activity, for example and in a non-limiting manner by direct or indirect interaction with the DNA sequence or the DNA and/or its assigned RNA sequence or mRNA and/or by direct or indirect modulation or interaction with the gene product in the form of the (poly)peptide encoded by the gene or by the DNA sequence. The inhibitor or repressor is in particular a substance having antiviral properties against, hepatitis viruses, in particular hepatitis C viruses.

In other words, the present invention aims essentially for a targeted, suppression of the gene expression or the gene activity of genes associated with the multiplication or replication or synthesis of hepatitis C viruses, in particular ISG15, which may, in a general manner, also be referred to as gene-knockdown or as gene-silencing.

With regard to the term “gene” used in accordance with the invention, in this respect the corresponding nucleic acid molecule or the corresponding DNA sequence is also included. However, the present invention likewise relates, as mentioned above, to the RNA sequence assigned to the gene, in particular mRNA, which, so to speak, acts as post-transcriptional product and, so to speak, may be complementary to the cociogenic strand of the DNA of the gene. Likewise, in the context of the present invention, it is also possible to use the (poly)peptide encoded by the nucleic acid or the gene, in particular as defined above,—and thus, as it were, the translation product—or be used as target molecule or target. The term “nucleic acid molecule” is synonymous with the term “polynucleic acid” or “polynucleic acid molecules” and may refer both to DNA and to RNA. In this context, the term “DNA sequence” or “RNA sequence” refers not only to the complete DNA assigned to or corresponding to the gene, but also to corresponding sections of the gene, or not only to complete RNA synthesized in particular in the context of transcription, but also to RNA sections or RNA fragments.

With regard to the gene taken into consideration in the context of the use according to the invention, this is preferably a human gene.

The gene taken into consideration in the context of the use according to the invention may in particular be an interferon-stimulated gene (ISG). In this respect, the gene may be a gene stimulated by interferons of type 1, preferably α-interferon and/or β-interferon. As described above, this is in particular a gene which can be induced or activated as a result of an increased interferon concentration, such that its expression is increased in particular under the control of interferon—caused, for example, by an infection with hepatitis C viruses.

In this respect, the applicant has found, in a completely surprising manner, that, the gene ISG15 forms a gene responsible with regard to the multiplication or replication of hepatitis C viruses or associated therewith. This is likewise an interferon-stimulated gene. The gene in question is in particular ISG15, in particular having the transcript ID (Locus) NM_{—}005101 or in particular in accordance with sequence protocol I and/or in particular in accordance with SEQUENCE LISTING. In this respect, the applicant has found, in a completely surprising manner, that ISG15, which is also referred to as Homo sapiens ISG15 Ubiquitin-like Modifier, is a particularly effective target with regard to the use according to the invention in the context of the therapy of hepatitis type C, the inactivation or suppression of which leads to a significant reduction of hepatitis C viruses in the host system.

The sequence protocol I mentioned above and the SEQUENCE LISTING listed above refer to the DNA sequence of the gene ISG15. Here, sequence protocol I is synonymous with or has the same meaning as the corresponding SEQUENCE LISTING. The essential difference between the sequence protocols is that sequence protocol I is based on a scientifically standardized notation or representation, whereas the SEQUENCE LISTING is based, on a notation or representation standardized according to patent law and generated using the software PatentIN Version 3.3. Thus, with respect to the DNA or with respect to the gene, the difference is purely formal in the presentation, but not in the content.

With regard to the inhibitor or repressor used in the context of the use according to the invention it should in this regard be a substance which reduces or neutralizes, in particular inhibits, the activity of the gene, in particular for ISG15.

In the context of the present invention, it is particularly advantageous for the inhibitor or the repressor to be a substance which interacts with the gene, in particular with ISG15, or with its DNA sequence and/or with its assigned RNA sequence and/or with its assigned (poly)peptide. In this respect, it is particularly advantageous for the substance to reduce and/or neutralize, in particular inhibit, the activity of the gene, in particular for ISG15, and/or its DNA sequence and/or its assigned RNA sequence and/or its assigned (poly)peptide.

In this context, the term “inhibitor” or “repressor” is to be understood in a very broad sense; it may in particular be a substance which interacts directly and/or indirectly, for example via metabolic cascades or signal transductions, with the appropriate target, in particular with ISG15, reducing or neutralizing, in particular inhibiting, its gene activity in the process. Here, the modulation of the gene, in particular of ISG15, or of the products (for example transcription and/or translation products) assigned to the gene or ISG15, may also be caused in the form of precursor substances which, for example, are converted only in the cell or host system into the actual interacting substance, for example via specific metabolic processes.

As discussed below, the direct and/or indirect interaction of the substance with the target structure, in particular with the gene, such as ISG15, may be realized on many levels:

According to a first preferred embodiment according to the invention, the interacting substance or the inhibitor and/or repressor may interact with the promoter and/or enhancer of the gene, in particular of ISG15, such that binding of in particular endogenous transcription factors, in particular activators, to the promoter and/or enhancer is prevented or at least inhibited.

However, it is likewise also possible for the substance or the inhibitor and/or repressor to interact with an in particular endogenous transcription factor, in particular activator, such that binding of the transcription factor, in particular activator, to the promoter and/or enhancer of the gene, in particular for ISG15, is prevented or at least inhibited.

However, it is likewise also possible for the substance or the inhibitor and/or repressor to interact with an in particular endogenous transcription factor, in particular activator, such that binding of the transcription factor, in particular activator, to the promoter and/or enhancer of the gene, in particular for ISG15, is prevented or at least inhibited. In this context, the inhibitor and/or repressor may interact with in particular endogenous mediators and/or factors regulated by the gene, in particular ISG15-regulated mediators and/or factors, and/or with in particular endogenous mediators and/or factors regulating the gene, in particular ISG-regulating mediators and/or factors, in particular such that the activity of the gene, in particular of ISG15, and/or its DNA sequence and/or its assigned RNA sequence and/or its assigned (poly)peptide is reduced and/or neutralized, in particular inhibited.

However, it is likewise also possible for the substance or the inhibitor and/or repressor to react with the endogenous transcription activators themselves, thus causing an inactivation of the transcription factor or activator per se, to reduce or neutralize, in particular inhibit, the gene activity, in particular for ISG15, in this manner.

The substance in the form of an inhibitor and/or repressor used in the context of the use according to the invention may, firstly, be a substance discovered on the basis of the process according to the invention, illustrated below, for identifying such substances.

According to an embodiment which is particularly preferred in accordance with the invention, the inhibitor and/or repressor may, secondly, be an RNA sequence which interacts preferably with the RNA sequence, in particular mRNA sequence, assigned, to the gene, in particular ISG15. According to this embodiment of the present invention, the inhibitor or repressor should be an RNA or RNA sequence which is in particular complementary to the RNA or mRNA or RNA sequence or mRNA sequence assigned to the gene.

In this connection, in the context of the present invention, it is particularly advantageous for the inhibitor and/or repressor to be an RNA sequence in the form of an oligomer, in particular having 15 to 20 bp (base pairs), preferably 18 to 25 bp, preferably 21 to 23 bp.

In the context of the present invention, the RNA sequence interacting with the RNA sequence assigned to the gene, in particular ISG15, is preferably a single-strand RNA, which, in this respect, should, in accordance with a particularly preferred embodiment, be an antisense strand in particular with respect to the RNA, in particular mRNA, assigned to the gene.

However, it is likewise also possible for the RNA sequence used according to the invention and interacting with the RNA sequence assigned, to the gene, in particular ISG15, to be a double-strand RNA which, in particular in the context of further modifications or metabolic processes, may be converted into or modified to a single-strand RNA, in particular in the form of an antisense strand, as defined above.

In accordance with a particularly preferred embodiment according to the invention, the inhibitor and/or repressor described above is an siRNA (small interfering RNA), where the siRNA is directed in particular against ISG15, in particular against ISG15-specific mRNA or against mRNA assigned to ISG15. In other words, the nature of the siRNA should be such that an interaction with the mRNA, in particular as defined above, is possible and, as a consequence, leads to the deactivation or to the degradation of the corresponding RNA. For example, the siRNA may be complementary to the mRNA or to sections of the mRNA. In this context, the applicant has found, in a completely surprising manner, that such an siRNA leads to particularly good results with regard to a reduction or neutralization of the gene activity of the gene, in particular ISG15, associated with the multiplication or replication of hepatitis C viruses.

The siRNA used in the context of the use according to the invention is, in accordance with a very particularly preferred embodiment, an siRNA which is directed against ISG15 and which can be obtained from Qiagen, Hilden, Germany, under the product number or order number SI00072337 (human) or SI01007531 (murine).

Without wishing to be bound to this theory, the mode of action of the specific ISG15-specific siRNA can be understood such that initially an siRNA/protein complex is formed in the cell or host system, which complex is also referred to as RNA-induced silencing complex (RISC), where a protein complex binds the antisense strand of the siRNA and cuts the complementary mRNA. In this context, the RISC complex has RNA helicase and RNA nuclease activities. In the interaction with the mRNA which, so to speak, represents the transcription product of the gene associated with the multiplication or replication of hepatitis C viruses, in particular ISG15, these properties lead to its unwinding and cleavage. As a consequence, the mRNA is degraded, which leads to a reduction of the translation of this mRNA and thus to gene-silencing or gene-knockout on the post-transcriptional level and thus to gene inactivation.

According to the invention, it may also be intended for the inhibitor and/or repressor to be an inhibitor or repressor which represents in particular the antisense strand of an siRNA. In the context of the present, invention, it may likewise be intended for the inhibitor or repressor to represent a complex of an antisense strand of the siRNA and at least one protein component, where this complex may in particular be an RISC complex (RISC=RNA-induced silencing complex).

A central concept of the present invention according to this aspect of the invention using an siRNA consists in the introduction of in particular synthetically prepared siRNA having a specificity for mRNA as transcription product, in particular of ISG15, into the cell or host system, which leads to a degradation of the mRNA of the target gene, i.e. in particular ISG15, which in turn leads to a reduction of the gene products and thus to gene-silencing or gene-knockout. The principle on which, according to this embodiment, the invention is based may also be referred to as RNA interference, on the basis of which the expression of certain (target) genes, in the present case in particular ISG15, is reduced. This is a result of in particular the interaction of siRNA directed specifically against a gene with the mRNA of the gene with the consequence of a degradation of the mRNA, which corresponds to a (gene) inactivation.

The application or the introduction of the siRNA in question into the cell or host system is known per se to the person skilled in the art so that this requires no further explanations. For example, the introduction or transfection may also take place via polyethyleneimine complexation (PEI complexation).

On application, the inhibitor or repressor employed according to the use according to the present invention should be administered in pharmaceutically effective amounts. In addition, the inhibitor or the repressor should be administered systemically, for example intravenously. The appropriate measures, too, are known as such to the person skilled in the art, so that this requires no further explanations.

In the context of the use according to the invention, it may likewise be intended for the inhibitor or repressor, in particular as described above, to be administered together with at least one interferon, in particular α-interferon, preferably pegylated α-interferon. In this context, a combination of the ISG15-specific siRNA described above or the siRNA directed against ISG15 with an interferon is of particular advantage. This is because the applicant observed—as shown below by the working examples—in a completely surprising manner a synergistic effect in the context of the combination, described above, of, firstly, inhibitor and/or repressor and, secondly, interferon with regard to the neutralization or reduction of multiplication or replication of hepatitis C viruses in the affected host systems or cell systems. Likewise, it is possible for the inhibitor and/or repressor to be administered together with and/or in combination with at least one substance having antiviral properties against hepatitis viruses, in particular hepatitis C viruses.

Altogether, in the context of the use according to the invention according to the present aspect of the invention, a novel way of treating a hepatitis C infection or hepatitis C disorders is provided, which is based on a completely novel approach, namely the targeted inactivation of a gene associated with the multiplication or replication of hepatitis C viruses, in particular ISG15.

The present invention furthermore provides—according to a second aspect of the present invention—the use of at least one substance, in particular an inhibitor and/or repressor, for preparing a medicament or pharmaceutical for the prophylactic and/or therapeutic treatment of hepatitis, in particular hepatitis type C, where the substance regulates, in particular at least reduces or inhibits, the gene activity and/or gene expression of at least one interferon-stimulated gene, in particular of ISG15.

For further explanations in respect of the use according to the invention according to the second aspect of the present invention, reference may be made to the explanations given for the previous aspect, which, in this respect, apply correspondingly.

In addition, the present invention furthermore provides—according to a third aspect of the present invention—a use of at least one interferon-stimulated nucleic acid molecule, in particular gene, preferably ISG15, and/or its DNA sequence and/or its assigned RNA sequence and/or at least one (poly)peptide encoded by the nucleic acid for identifying and/or providing a pharmaceutical for the prophylactic and/or curative treatment of hepatitis, in particular hepatitis type C, and/or for predicting individual effects of the pharmaceutical and/or side effects of the pharmaceutical.

With regard to the use in accordance with this aspect according to the invention, the gene associated with the multiplication or replication of hepatitis C viruses, in particular ISG15, can in a way be used as starting object for identifying or providing pharmaceuticals with respect to a hepatitis C infection or disorder. Here, the pharmaceuticals may be of a nature such that—as indicated above—they interact directly or indirectly with the gene defined above or the RNA, in particular mRNA, assigned thereto, and/or the corresponding (poly)peptide, in this manner leading, in particular via reduction or neutralization, in particular inhibition, of the gene activity, to a reduction or neutralization of the multiplication or replication of hepatitis C viruses and thus to amelioration or cure of the hepatitis C disorder. Accordingly, these may be substances having a gene-regulatory action.

The person skilled in the art is essentially familiar with the principles with respect to the specific area of application of the use according to the invention according to this aspect of the invention, so that this does not require any further explanations.

The present invention furthermore provides—in accordance with a fourth aspect of the present invention—a process for identifying an inhibitor and/or repressor of an interferon-stimulated nucleic acid molecule, in particular gene, preferably of ISG15, and/or its DNA sequence and/or its assigned RNA sequence, which comprises the following steps:
(a) bringing the nucleic acid molecule into contact with at least one test substance under conditions allowing an interaction, in particular binding, of the test substance(s) with the nucleic acid molecule; and
(b) detection and/or analysis of whether the test substance(s) limits or neutralizes the gene activity and/or expression of the nucleic acid molecule and/or of whether the test substance(s) limits or neutralizes multiplication and/or replication of hepatitis viruses, in particular hepatitis C viruses.

The process according to the invention may be carried out, for example, in vitro, where in a way an interaction of the test substance with the nucleic acid molecule or the gene is studied and, if an interaction is present, a resulting reduced gene activity as a consequence of this interaction may be inferred. The process according to the invention may likewise be carried out in an appropriate host system, where the host should be a carrier of the gene associated with the multiplication or replication of hepatitis C viruses, in particular ISG15, and advantageously also have a corresponding expression system. The interaction or the modulated gene activity can be demonstrated using processes known per se to the person skilled in the art.

In this context, the present invention—according to a fifth aspect of the present invention—relates to a process for identifying an inhibitor and/or repressor of a (poly)peptide encoded by an interferon-stimulated nucleic acid molecule, in particular gene, preferably ISG15, and/or by its DNA sequence and/or by its assigned RNA sequence, which comprises the following steps:
(a) bringing the (poly)peptide into contact with at least one test substance under conditions allowing an interaction, in particular binding, of the test substance(s) with the (poly)peptide; and
(b) detection and/or analysis of whether the test substance(s) limits or neutralizes the activity of the (poly)peptide and/or of whether the test substance(s) limits or neutralizes the multiplication and/or replication of hepatitis viruses, in particular hepatitis C viruses.

According to this aspect, the process according to the invention is thus focused on modulating the gene product, in particular in the form of a protein or (poly)peptide. The process can be carried out in a manner known per se to the person skilled in the art, both in vitro and in vivo, in a host system, where in the latter case the host should preferably carry the nucleic acid coding for the (poly)peptide to be examined. However, an interaction may likewise also be carried out in vitro using isolated (poly)peptides.

In addition, the present invention likewise relates to a process for identifying a substance which interacts with an in particular endogenous mediator and/or factor which is regulated by an interferon-stimulated nucleic acid molecule, in particular gene, preferably ISG15, or which regulates an interferon-stimulated nucleic acid molecule, in particular gene, preferably ISG15, where the process comprises the following steps:
(a) bringing the mediator and/or factor into contact with at least one test substance under conditions allowing an interaction, in particular binding, of the test substance(s) with the mediator and/or factor; and
(b) detection and/or analysis of whether the test substance(s) modulates the activity of the mediator and/or factor and/or of whether the test substance (s) limits or neutralizes the multiplication and/or replication of hepatitis viruses, in particular hepatitis C viruses.

According to the process mentioned above, the interacting substance is preferably an inhibitor and/or repressor of the mediator and/or factor, if this is associated with an activation of the interferon-stimulated nucleic acid molecule, in particular gene, preferably ISG15. If the mediator and/or factor itself is associated with an inactivation or repression of the interferon-stimulated nucleic acid molecule, in particular gene, preferably ISG15, the interacting substance is, with respect to the mediator and/or factor, an activator—however, with regard to the interferon-stimulated nucleic acid molecule, in particular gene, preferably ISG15, based on the above definition, such a substance is likewise an inhibitor or repressor, since in this case, too, the final result is an inactivation or repression of the interferon-stimulated nucleic acid molecule, in particular gene, preferably ISG15.

The processes according to the invention in accordance with the fourth and fifth aspect of the present invention can be carried out such that a plurality of test substances are used and the following steps are carried out:
(a) testing various test substances in various reaction vessels, where the test substances which do not limit or neutralize the gene activity and/or expression and/or activity of the nucleic acid molecule (DNA or RNA, in particular mRNA) and/or which do not limit or neutralize the activity of the (poly)peptide and/or which do not limit or neutralize the multiplication and/or replication of hepatitis viruses, in particular hepatitis C viruses and/or which do not interact with the mediator and/or factor, are no longer taken into consideration in the further test process;
(b) distribution of test substances from those reaction vessels where, in step (a), a reduction or neutralization of the gene activity and/or expression and/or activity of the nucleic acid molecule has been determined and/or a reduction or neutralization of the activity of the (poly)peptide has been determined and/or a reduction or neutralization of the multiplication and/or replication of hepatitis viruses, in particular hepatitis C viruses has been determined and/or an interaction with the mediator and/or factor has been determined, to new reaction vessels and repetition of step (a) with the new reaction vessels; and
(c) repetition of step (b) until a single test substance has been identified to which the reduction or neutralization of the gene activity and/or expression of the nucleic acid molecule and/or the reduction or neutralization of the activity of the (poly)peptide and/or the reduction or neutralization of the multiplication and/or replication of hepatitis viruses, in particular hepatitis C viruses, can be assigned and/or to which the interaction with the mediator and/or factor can be assigned.

Here, it is likewise possible to carry out the processes according to the fourth and the fifth aspect of the present invention such that the test substance(s), the nucleic acid molecule (DMA or RNA, in particular mRNA) and/or the (poly)peptide are coupled to a readout system and/or that a readout system is added to the test batch and/or that the readout system affords, after binding of the test substance (s) to the nucleic acid molecule and/or the (poly)peptide, a detectable signal.

In the context of the processes mentioned above, the test substances may be low-molecular-weight substances, peptides, aptamers, antibodies, DNA, RNA, in particular siRNA, and/or fragments or derivatives thereof.

As explained above, the processes mentioned above can be carried out, for example, in a host or host system, where the host or the host system preferably comprises the genes defined above and also has a corresponding expression system. Such hosts are known per se to the person skilled in the art, or, against the background of the present invention, the person skilled in the art is at any time able to select specific host systems, so that this does not require any further explanations. The processes according to the invention can likewise be carried out in the form of high-throughput processes and/or in a computer-assisted manner.

For further details with respect to the processes according to the invention in accordance with the fourth and the fifth aspect of the present invention, reference may be made to the explanations given for the other aspects of subject matters of the present invention, which, in this respect, apply correspondingly.

In addition, the present invention furthermore provides—according to a sixth aspect of the present invention—a process for improving the pharmacological properties of the test substances identified by the process according to the fourth and/or fifth aspect of the present invention, where
(a) the binding site of the test substance to the nucleic acid molecule (DNA or RNA, in particular mRNA) or to the (poly)peptide and, if appropriate, the binding site of the nucleic acid molecule or the (poly)peptide to the test substance are identified;
(b) the binding site of the test substance and the nucleic acid molecule or the (poly)peptide is modified by molecular modeling; and
(c) the test substance is modified such that its binding specificity or binding affinity or binding avidity for the nucleic acid molecule or the (poly)peptide is increased.

In this respect, the binding site in step (a) can be identified by site-specific mutagenesis, the relevant processes being known per se to the person skilled in the art.

In addition, the present invention furthermore provides—in accordance with a seventh aspect of the present invention—a process for modifying a test substance identified or improved by the processes according to the fourth and/or fifth and/or sixth aspect of the present invention, where the test substance as lead structure is modified further to achieve
(i) a modified active center, a modified activity spectrum and/or a modified organ specificity and/or
(ii) an improved activity and/or
(iii) a reduced toxicity (an improved therapeutic index) and/or
(iv) reduced side effects and/or
(v) onset of the therapeutical activity at a different time and/or different length of therapeutic activity and/or
(vi) changed pharmacokinetic parameters (in particular bioabsorption, distribution, metabolism and/or excretion) and/or
(vii) modified physicochemical parameters, in particular solubility, hygroscopic properties, color, taste, smell, stability and/or state, and/or
(viii) improved general specificity, organ/tissue specificity, and/or
(ix) optimized administration form and/or route, in particular by
   (a) esterification of carboxyl groups and/or
   (b) esterification of hydroxyl groups with carboxylic acids and/or
   (c) esterification of hydroxyl groups, in particular to phosphates, pyrophosphates or sulfates and/or hemisuccinates and/or
   (d) formation of pharmaceutically acceptable salts and/or
   (e) formation of pharmaceutically acceptable complexes and/or
   (f) synthesis of pharmacologically active polymers and/or
   (g) introduction of hydrophilic groups and/or
   (h) introduction and/or exchange of substituents in aromatics and/or side chains and/or modification of the substituent pattern and/or
   (i) modification by introduction of isosteric and/or bioisosteric groups and/or
   (j) synthesis of homologous compounds and/or
   (k) introduction of branched side chains and/or
   (l) conversion of alkyl substituents into cyclic analogs and/or
   (m) derivitization of hydroxyl groups to ketals and/or acetals and/or
   (n) N-acetylation to amides and/or phenylcarbamates and/or
   (o) synthesis of Mannich bases and/or imines and/or
   (p) conversion of ketones and/or aldehydes into Schiff bases, oximes, acetals, ketals, enol esters, oxazolidines, thiozolidines or combinations thereof.

Here, it is possible in the context of the process according to the invention for the identified, improved or modified test substance, in particular the inhibitor and/or repressor of the gene(s) mentioned above, to be further improved pharmacologically by peptidomimetics.

For further explanations in respect to the processes according to the invention in accordance with this aspect of the present invention, reference may be made to the explanations for the above aspects of the present invention, which apply correspondingly.

Additionally, the present invention furthermore provides—according to an eighth aspect of the present invention—a process for identifying and/or determining at least one nucleic acid, molecule, in particular gene, preferably human and/or interferon-stimulated gene, associated with the replication of hepatitis viruses, in particular hepatitis C viruses, where the process comprises the following steps:
(a) generation of a gene expression and/or gene activity profile of a large number of subjects of a collective of subjects, where (i) the subjects of a first group of the collective of subjects are infected by hepatitis viruses, in particular hepatitis C viruses, and (ii) the subjects of a second group of the collective of subjects do not have such an infection;
(b) analysis and comparison or aligning of the respective gene expression and/or gene activity profiles of (i) subjects of the first group and (ii) subjects of the second group and
(c) identification of at least one nucleic acid molecule, in particular at least one gene, which, in (i) the subjects of the first group, compared to (ii) the subjects of the second group, has increased gene expression and/or gene activity.

The process may, subsequent to step (c), comprise the following step (d):
(d) assignment of the nucleic acid molecule, in particular gene, identified in step (c), as a nucleic acid molecule, in particular gene, associated with the multiplication and/or replication of hepatitis viruses, in particular hepatitis C viruses.

The process according to the invention according to this aspect of the present invention can be employed, for example, by a differential expression using DNA chips. Here, the procedure adopted may be such, for example, that initially RNA is isolated from the blood of a subject to be examined, and this isolate is added onto specific gene chips and, in evaluation or analysis processes known per se to the person skilled in the art, the degree of expression of certain genes is determined for subjects having a hepatitis C infection in comparison to subjects without hepatitis C infection, and a gene, in particular an interferon-stimulated gene, having an increased degree of expression is assigned the properties of a gene associated with the multiplication or replication of hepatitis C viruses, in particular a gene promoting the multiplication or replication of hepatitis C viruses.

In this manner, it is possible to identify further genes, in particular interferon-stimulated genes, which are associated with the multiplication or replication of hepatitis C viruses in a hepatitis C infection or hepatitis C disorder.

The present invention furthermore relates—according to a ninth aspect of the present invention—to a pharmaceutical composition, preferably for the prophylactic or therapeutic treatment of hepatitis C disorders, comprising effective, in particular pharmaceutically effective, amounts of at least one pharmacologically active substance, in particular an inhibitor and/or repressor, for a nucleic acid molecule, in particular gene, associated with the multiplication and/or replication of hepatitis viruses, in particular hepatitis C viruses, preferably ISG15, and/or for its DNA sequence and/or for its assigned RNA sequence, in particular for its assigned mRNA sequence, and/or for its assigned (poly)peptide, the substance being obtainable by the process according to the fourth and/or fifth and/or sixth aspect of the present invention.

For further relevant details in respect of the pharmacologically active substance according to the invention, reference may be made to the other explanations of the further aspects of the present invention, which apply correspondingly in this context.

In addition, the present invention furthermore provides—in accordance with a tenth aspect of the present invention—a pharmaceutical composition, preferably for the prophylactic or therapeutic treatment of hepatitis C disorders, comprising effective, in particular pharmaceutically effective, amounts of at least one siRNA, where the siRNA regulates, in particular reduces or at least inhibits, the gene activity and/or gene expression of at least one interferon-stimulated gene, in particular of ISG15.

The siRNA used in the context of the pharmaceutical composition according to the invention is, according to a very particularly preferred embodiment, an siRNA which is directed against ISG15 and which can be obtained from Qiagen, Hilden, Germany, under the product, number or order number SI00072387.

For further details with respect to the pharmaceutical composition according to the invention in accordance with this inventive aspect of the present invention, reference may be made to the explanations given for the other above-mentioned aspects of the present invention, which, in this context, apply correspondingly.

Further developments, modifications and variations of the present invention will be directly evident and achievable for the person skilled in the art reading the description without departing from the scope of the present invention.

The present invention is illustrated by means of the following working examples which do not, however, restrict the present invention in any way.

### WORKING EXAMPLES

### Methods and Test Results:

1. Isolation of Total RNA

For the extraction of total RNA, the cells were covered with 500 μl of Trizol. Using a scraper made of plastic, the adhering cells were detached from the base and transferred into a reaction vessel, 0.1 ml of chloroform/1 ml of Trizol was added and mixed in by shaking. Centrifugation at 12,000 g and at 2 to 8° C. for 15 min resulted in phase separation of the phenol/chloroform mixture. The aqueous phase was removed and the dissolved RNA was precipitated using 0.5 ml of isopropanol/1 ml of Trizol. The pellet was then washed with 75% ethanol, dried under reduced pressure and then dissolved in RNase-free water. The RNA was subsequently purified using the “RNeasy Mini” kit (Qiagen, Hilden, Germany) in accordance with the instructions of the manufacturer and stored at −20° C. until further analysis.

2. Quantitative Real-Time PCR:

Reverse transcription of RNA followed by a polymerase chain reaction (RT-PCR) is a sensitive method for the quantification of specific mRNAs. In a one-step RT-PCR process (one-step RT-PCR), using specific primers, initially, the mRNA of the wanted gene is transcribed into complementary DNA (cDNA) which in turn affords the template or the basis for the PCR that follows.

To be able to make quantitative statements with regard to the amount of mRNA employed, the LightCycler Rotor-Gene 2000 from Corbett (Mortlake, Australia) was used. Via a fluorimeter component, the LightCycler measures the fluorescence of the fluorophore after binding to double-stranded DNA. Use was made of the QuantiTect SYBR Green RT-PCR Kit from QIAGEN; a 25-μl batch was, using a pipette, composed as follows: 5.25 μl of H₂O (RNase-free), 12.5 μl of SYBR Green RT-PCR Master Mix, 0.25 μl of QuantiTect RT-Mix, 2.5 μl of each primer (0.5 mM) and 2 μl of total RNA (100 ng to 200 ng). The LightCycler program used started with a 30-minute RT step at 55° C., followed by a 15-minute heat deactivation of the RT polymerase and a subsequent conventional PCR scheme, i.e. per cycle 5 s of denaturation at 95° C., 10 s of annealing temperature (55° C.) and 30 s of elongation at 72° C. Product formation was determined after each replication cycle via the increase in fluorescence. After on average 40 replication cycles, the melting curves of the products formed were measured to check the specificity of the PCR reaction. Owing to the melting properties of DNA, the fluorescence decreases with increasing temperature. The maximum change in fluorescence per temperature increase yields a maximum in the melting curve characteristic for each PCR product. The copy numbers, calculated by the LightCycler, of the measured genes were aligned with the housekeeping gene β-aktin and analyzed.

3. Suppression of the Gene Expression of ISG15 by siRNA:

The expression of ISG15 was switched off on the cell culture level in the HCV replicon system to examine the direct effect on HCV replication. Suppression of gene expression, also referred to as gene-knockdown or gene-knockout, is carried out using siRNA, via a cellular mechanism of processing. The dicer enzyme complex cleaves cell-atypical dsRNA into 21- to 23-bp-oligomers, the so-called small interfering RNAs (siRNA). The siRNA and a protein complex may form the RNA-induced silencing complex (RISC); this binds the antisense strand of the siRNA and cuts the complementary mRNA. The degration of the mRNA results in a reduction of the translation of these mRNAs and thus a gene-silencing on the post-transcriptional level.

A con1 replicon system based on the human hepatoma cell line HuH-7 and the murine MH1 cells, which likewise contain an HCV replicon, were, in a 96-well plate format, transfected with siRNAs directed against ISG15 (Qiagen) (human: order No. SI00072387, murine: order No. SI01007531). The effect of the gene suppression on the HCV replication was then examined.

To this end, in a reverse transfection batch, initially 12.5 ng of the siRNAs (5 nM) were dissolved in 3 μl of suspension buffer and, using a pipette, initially charged in the wells of the well plate. In a further batch, in each case 2.5 ng of the siRNAs were used for simultaneous silencing of further genes (not shown in the graphics). The control used was a non-codogenic siRNA. Per batch, 0.75 μl of the transfection reagent Hi-PerFect™ (Qiagen, Hilden, Germany) was then transferred into 24.25 μl of serum-free culture medium, and reagent and medium were mixed and added to the siRNAs initially charged. The transfection batch was then incubated at room temperature for 10 minutes. 1.10⁴ cells were then added to the transfection batches, the volume was made up to 200 μl with culture medium and the cells were incubated at 37° C., 5% CO₂ and saturated atmospheric humidity for 12 hours. The RNA was extracted as already described, and the decrease of the gene expression of ISG15 and the HCV replication were examined by quantitative RT-PCR and Westernblot.

4. Inhibition of HCV Replication during Extended Testing over 3 Months—Lack of Induction of Resistant Mutants:

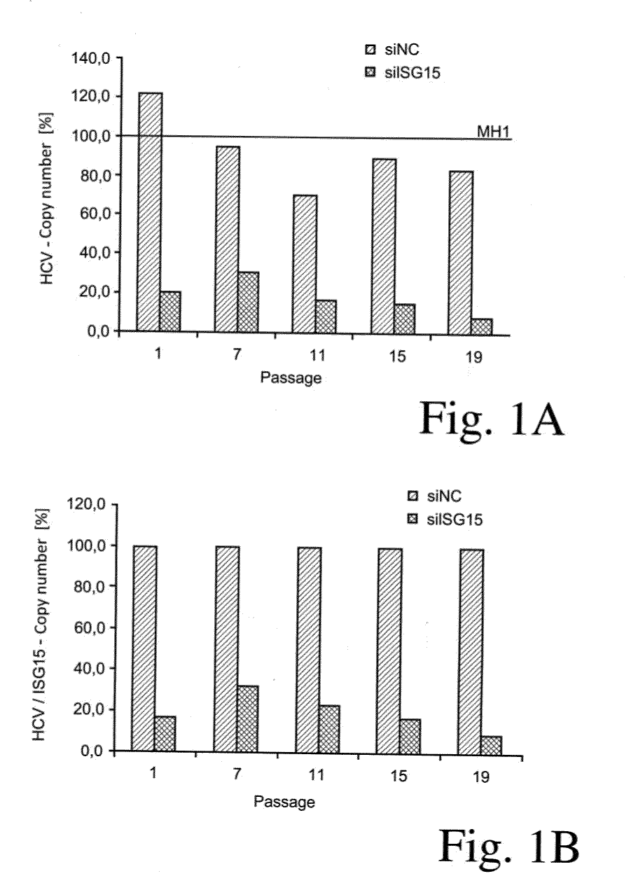

Murine MH1 HCV replicon cells were cultivated over 19 cell passages with non-coding siRNA or siRNA directed against ISG15. The HCV copy number/100,000 copies of β-aktin was determined for cell passages 1, 7, 11, 15 and 19 and in each case compared to the untreated control (100%). Since, during the entire duration of the experiment, the siRNA directed, against ISG15 maintained its anti-HCV activity, it has to be assumed that no mutants resistant to this batch nave been formed or were selected. FIG. 1A and FIG. 1B illustrate the results obtained (with siNC=non-silencing control, siISG15=ISG15—specific siRNA). In FIG. 1A, the HCV copy number has been normalized and compared with the untreated control (MH1), and in FIG. 1B the HCV/ISG15 copy number has been normalized and compared with the siNC control. FIG. 1A and FIG. 1B show the marked decrease of the HCV copy number on treatment with ISG15-specific siRNA (siISG15). As a consequence, the multiplication and replication under the action of siISG15 is reduced, too.

5. Inhibition of HCV NS5A Protein Expression by siRNA Knockdown of ISG15-synergism with IFN-α:

The murine MH1 and the human con1 HCV replicon cells were sowed in 6-well plates and incubated at 37° C. and an atmospheric CO₂ content of 5% to a confluence of 30 to 40%. Transfection with 5 nM siRNA (siNC=non-silencing control, siISG15=ISG15-specific human and murine siRNA) was carried out with “HiPerFect™ Transfection Reagent” (Qiagen, Hilden, Germany). After 8 h of incubation (37° C., 5% CO₂), the cells were washed and taken up again in culture medium and medium to which IFN-α had been added and incubated for a further 64 h. Subsequently, the cells were lyzed and a protein extraction was carried out. Using Westernblot, it was possible to detect the viral protein NS5A and the housekeeping gene β-aktin.

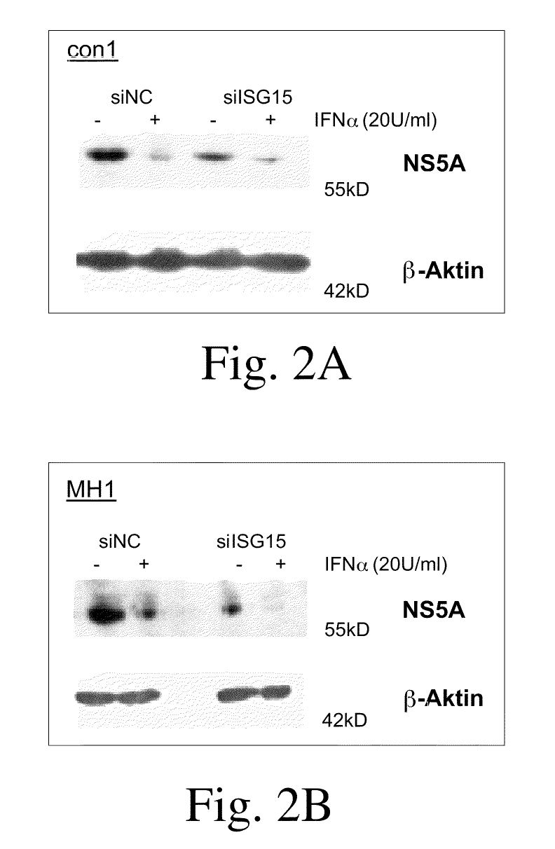

In the context of a Westernblot, FIG. 2A and FIG. 2B show that treatment with siRNA directed against ISG15 leads to a marked suppression of the HCV NS5A protein both in murine and in human HCV replicon systems. Furthermore, FIG. 2A and FIG. 2B show that synergism with IFN-α exists, since the additional administration of IFN-α (cf. FIG. 2A and FIG. 2B: +IFN-α) results in a significant reduction of NS5A synthesis compared to the batch which had not been treated with IFN-α (cf. FIG. 2A and FIG. 2B: −IFN-α).

FIG. 1A shows the inhibition of the HCV replication and the lack of induction of resistant, mutants on treatment with ISG15-specific siRNA (siISG15) compared to a treatment with siNC, where the HCV copy number has been normalized against the untreated control (MH1) and compared.

FIG. 1B shows the inhibition of the HCV replication and the lack of induction of resistant mutants on treatment with ISG15-specific siRNA (siISG15) compared to a treatment with siNC, where the HCV/ISG15 copy number has been normalized against the siNC control and compared.

FIG. 2A shows, by way of a Westernblot, the results of a treatment, carried out on human con1 HCV replicon cells, with siRNA directed against ISG15, firstly without additional administration of IFN-α (−IFN-α) and secondly with additional administration of IFN-α (+IFN-α).

FIG. 2B shows, by way of a Westernblot, the results of a treatment, carried out on murine MH1 HCV replicon cells, with siRNA directed against ISG15, firstly without additional administration of IFN-α (−IFN-α) and secondly with additional administration of IFN-α (+IFN-α).

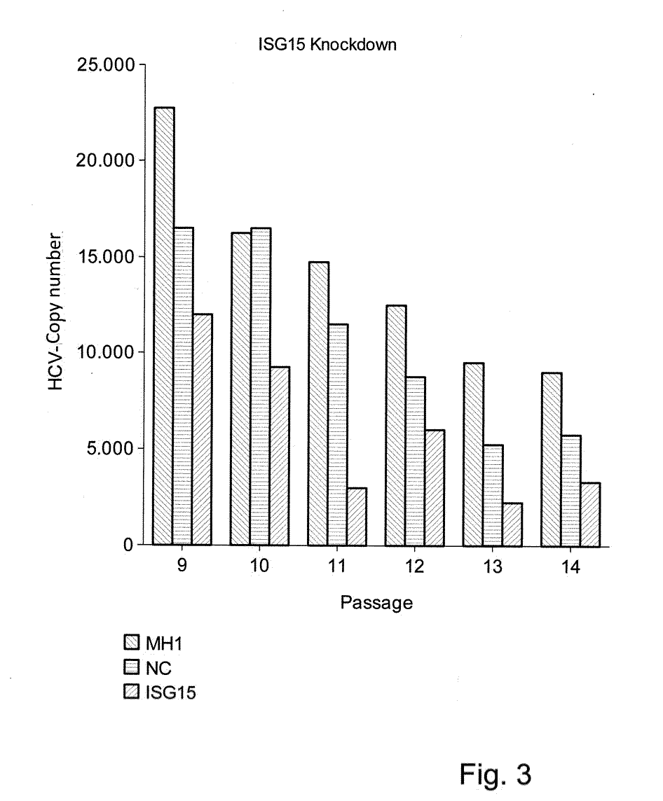

FIG. 3 shows a comparison of the replication of hepatitis C viruses in MH1 cells for various passages, where the HCV copy number is based on 100,000 β-aktin molecules (MH1=untreated control, NC=non-silencing control, ISG15=ISG15-specific siRNA), For all passages, when ISG15-specific siRNA is used, a marked decline of the HCV copy number and thus a reduction of virus synthesis and replication can be observed compared to MH1 and NC.

1. A method of treating a human suffering from hepatitis, said method comprising: using or administering, in an amount therapeutically efficient, an inhibitor and/or repressor of a nucleic acid molecule associated with the multiplication and/or replication of hepatitis viruses and/or of its DNA sequence and/or of its assigned RNA sequence and/or of its assigned (poly)peptide.

2. The method as claimed in claim 1, where the gene is a human gene.

3. The method as claimed in claim 1, where the gene is an interferon-stimulated gene selected from a gene stimulated by interferons of type I, α-interferon and/or β-interferon.

4. The method as claimed in claim 1, where the gene is ISG15 having the transcript ID (Locus) NM_{—}005101 and/or according to Sequence Protocol I and/or according to SEQUENCE LISTING.

5. The method as claimed in claim 1, where the inhibitor and/or repressor is a substance which reduces and/or neutralizes the activity of ISG15.

6. The method as claimed in claim 1, where the inhibitor and/or repressor is a substance which interacts with ISG15 and/or with its DNA sequence and/or with its assigned RNA sequence and/or with its assigned (poly)peptide.

7. The method as claimed in claim 1, where the inhibitor and/or repressor is an RNA sequence which interacts with the RNA sequence assigned to the gene, where the inhibitor and/or repressor is an RNA sequence in the form of an oligomer having 15 to 20 base pairs (bp).

8. The method as claimed in claim 1, where the inhibitor and/or repressor is administered together with at least one interferon and/or in combination with at least one substance displaying antiviral properties to hepatitis viruses.

9. A process for identifying an inhibitor and/or repressor of an interferon-stimulated nucleic acid molecule and/or its DNA sequence and/or its assigned RNA sequence, which comprises the following steps:
(a) bringing the nucleic acid, molecule into contact with at least one test substance under conditions allowing an interaction of the test substance(s) with the nucleic acid molecule; and
(b) detection and/or analysis of whether the test substance(s) limits or neutralizes the gene activity and/or expression of the nucleic acid molecule and/or of whether the test substance(s) limits or neutralizes the multiplication and/or replication of hepatitis viruses.

10. The process as claimed in claim 9, where a plurality of test substances is used and the following steps are carried out:
(a) testing various test substances in various reaction vessels, where the test substances which do not limit or neutralize the gene activity and/or expression and/or activity of the nucleic acid molecule and/or which do not limit or neutralize the activity of the (poly)peptide and/or which do not limit or neutralize the multiplication and/or replication of hepatitis viruses are no longer taken into consideration in the further test process;
(b) distribution of test substances from those reaction vessels where, in step (a), a reduction or neutralization of the gene activity and/or expression and/or activity of the nucleic acid molecule has been determined and/or a reduction or neutralization of the activity of the (poly)peptide has been determined and/or a reduction or neutralization of the multiplication and/or replication of hepatitis viruses has been determined, to new reaction vessels and repetition of step (a) with the new reaction vessels; and
(c) repetition of step (b) until a single test substance is identified, to which the reduction or neutralization of the gene activity and/or expression of the nucleic acid molecule and/or the reduction or neutralization of the activity of the (poly)peptide and/or the reduction or neutralization of the multiplication and/or replication of hepatitis viruses can be assigned.

11. A process for identifying and/or determining at least one nucleic acid molecule associated with the replication of hepatitis viruses, where the process comprises the following steps:
(a) generation of a gene expression and/or gene activity profile of a large number of subjects of a collective of subjects, where (i) the subjects of a first group of the collective of subjects are infected by hepatitis viruses, and (ii) the subjects of a second group of the collective of subjects do not nave such an infection;
(b) analysis and comparison or aligning of the respective gene expression and/or gene activity profiles of (i) subjects of the first group and (ii) subjects of the second group; and
(c) identification of at least one nucleic acid molecule which, in (i) the subjects of the first group, compared to (ii) the subjects of the second group, has increased gene expression and/or gene activity.

12. The process as claimed in claim 11, where the process, subsequent to step (c), comprises the following step:
(d) assignment of the nucleic acid molecule identified in step (c) as a nucleic acid molecule associated with the multiplication and/or replication of hepatitis viruses.

13. A pharmaceutical composition for the therapeutic treatment of hepatitis disorders, comprising a pharmaceutically effective amount of at least one pharmacologically active substance selected from an inhibitor and/or repressor for a nucleic acid molecule associated with the multiplication and/or replication of hepatitis viruses and/or for its DNA sequence and/or for its assigned RNA sequence and/or for its assigned (poly)peptide, wherein the substance is obtained by the process as claimed in claim 9.

14. The pharmaceutical composition of claim 13 comprising a pharmaceutically effective amount of at least one siRNA, where the siRNA reduces or at least inhibits the gene activity and/or gene expression of at least one interferon-stimulated gene.


SEQUENCE LISTING (2 sequences)

SEQ ID NO: 1 - DNA, 634 bp, Homo sapiens
cggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagattggcgtgcacgccttccagcagcgtctggctgtccacccgagcggtgtggcgctgcaggacagggtcccccttgccagccagggcctgggccctggcagcacggtcctgctggtggtggacaaatgcgacgaacctctgagcatcctggtgaggaataacaagggccgcagcagcacctacgaggtccggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaagagaat

SEQ ID NO: 2 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS